(19) United States
(12) Patent Application Publication
(10) Pub. No.: US 2025/0009342 A1
(43) Pub. Date: Jan. 9, 2025

(54) SYSTEMS AND METHODS FOR ROTATIONAL ULTRASOUND FOCUSING

(71) Applicant: LUMA Vision Limited, Dublin (IE)

(72) Inventors: Jakob Weiss, Munchen (DE); Christoph Hennersperger, Munchen (DE)

(21) Appl. No.: 18/763,044

(22) Filed: Jul. 3, 2024

Related U.S. Application Data
(60) Provisional application No. 63/525,259, filed on Jul. 6, 2023.

(51) Int. Cl. A61B 8/08; A61B 8/00; A61B 8/12; G01S 7/52; G01S 15/89

(52) U.S. Cl. CPC A61B 8/5207; A61B 8/12; A61B 8/4488; A61B 8/469; A61B 8/483; G01S 7/52085; G01S 15/8993; A61B 8/4461

(57) ABSTRACT

The present invention is directed to systems and methods for use in ultrasound imaging for selective optimization of imaging parameters for anatomical regions of interest. Systems and methods of the invention provide for selectively adjusting the imaging parameters to achieve a higher resolution and/or deeper penetration into tissue to thereby improve image quality and/or the field of view in a selected region of interest.

### CROSS-REFERENCE TO RELATED APPLICATIONS

This application claims priority to, and the benefit of, U.S. Provisional Application No. 63/525,259, filed Jul. 6, 2023, the content of which is incorporated by reference herein in its entirety.

### FIELD OF THE INVENTION

The invention generally relates to ultrasound imaging, and, more particularly, to systems and devices providing rotational focusing for selective optimization of imaging parameters for anatomical regions of interest.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image.

Ultrasound imaging can help a physician evaluate, diagnose and treat various medical conditions. When making a diagnosis based on an ultrasound examination, physicians must rely on adequate image quality, acquisition of proper views, and sufficient quantification of all relevant structures and flows.

For example, catheter-based endovascular ultrasound imaging technology employed within the vasculature (e.g. intravascular ultrasound (IVUS) or intracardiac echocardiography (ICE)) is commonly performed with two-dimensional (2D) ultrasound imaging. In IVUS/ICE imaging systems, an ultrasonic transducer assembly is attached to a distal end of a catheter. The catheter is carefully maneuvered through a patient's body to an area of interest, such as within a coronary artery (for the case of IVUS), or within the right atrium (for the case of ICE). The transducer assembly transmits ultrasound waves and receives echoes from those waves. The received echoes are then converted to electrical signals and transmitted to processing equipment, in which a resulting ultrasound image of the area of interest may be displayed.

Conventional 2D ultrasound imaging has been widely used because it can dynamically display 2D images of the region of interest in real-time. While 2D endovascular ultrasound is the standard of care, it requires the operator to know the anatomy at hand for navigation. This requires a high amount of dexterity in maneuvering the catheter image plane to visualize the target structure for a specific interventional use-case. Thus, both the catheter and the imaging plane must be concurrently maneuvered. 2D imaging is also limited to displaying a slice of the anatomy only.

Furthermore, in typical ultrasound systems configured to visualize inner body regions, dynamic forces are often employed, resulting in a dynamic movement of the body regions over time. These dynamic forces and movements make it difficult to stabilize internal imaging devices and to generate consistent and accurate images if imaging of the structure cannot be enabled in real-time (e.g., >20 Hz). As a result, the captured images often lack the necessary quality required to prescribe appropriate treatment or therapy. Because of the dynamic forces and movements in play, internal real-time imaging is limited to small two-dimensional areas or, as noted below, three-dimensional volumetric regions respectively.

2D array transducers have enabled three-dimensional (3D) ultrasound imaging. 3D ultrasound imaging was developed to address the drawbacks of 2D ultrasound imaging and to help diagnosticians and interventionalists acquire a full understanding of the spatial anatomic relationship. In particular, physicians can view an arbitrary plane of the reconstructed 3D volume, as well as panoramic view of the region of interest. Thus, 3D imaging can yield a superior depiction of target structures as well as evaluation, e.g. volumetric assessment.

However, 3D imaging systems have drawbacks and limitations. For example, 3D imaging systems provide a view of the region of interest that is limited to a pyramidical volume (e.g., a trapezoid fan angle that is either side- or forward-looking from the catheter), which is further limited to a 90-degree by 60-degree sector opening for advanced imaging catheters. As such, while 2D array transducers have enabled 3D ultrasound imaging, difficult engineering tradeoffs still exist between system complexity and achievable image quality. Thus, while 3D ultrasound imaging offers significant promise for a wide range of clinical applications, clinical impact is currently limited, in part because image quality is often inferior to 2D imaging using linear- or phased-array transducers. Imaging fully around a catheter in a 360-degree field of view with selective focusing can overcome the limitations of the 2D and 3D image quality as described above, and thus enable clinical users delivering better therapy.

### SUMMARY

The present invention recognizes the drawbacks of current ultrasound imaging systems, particularly those systems using an ultrasound probe for 3D imaging. In particular, current 2D and 3D ultrasound systems are limited in that they apply one generalized beamforming and reconstruction approach to the entire volume at hand. As a result, such systems suffer from associated technical challenges that limit the imaging quality of specific anatomical regions of interest within the full 360-degree view.

For example, due to processing constraints, current circumferential 3D ultrasound imaging utilizes a constant rotational velocity and a constant firing rate, which ultimately results in a constant angular resolution around the circumference. Thus, while circumferential 3D ultrasound imaging, via a rotating transducer or 2D array electronic scanning, allows for obtaining a rotationally symmetric imaging volume, the angular resolution for the entire volume depends on, among other factors, the constant firing rate and constant rotational speed. Further, in circumferential 3D ultrasound imaging via a rotating transducer, and also in 2D matrix array systems, the maximum transmit/receive firing rate is constrained by the electronic design, which, specifically for matrix array transducers, often requires micro-beamforming steps directly in the transducer in order to reduce data rates to the system.

The systems and methods of the present invention solve such problems by providing for live ultrasound imaging using a rotating transducer array or a 3D ultrasound transducer to selectively improve or adapt the imaging quality in specific directions of a region of interest. In particular, the present invention recognizes that imaging specific anatomical structures that lie in a particular angular direction may be improved if imaging parameters can be selectively adjusted to vary the imaging quality. Accordingly, the systems and methods of the present invention allow for selective directional focusing, depth adjustment, and/or resolution imaging, thereby allowing for selective optimization of imaging parameters for anatomical regions of interest.

For example, systems and methods of the invention provide for selectively adjusting the imaging settings to achieve, for example, a higher resolution, and/or a higher imaging depth, to ultimately improve imaging quality in an anatomical region of interest. Imaging the anatomical region of interest may benefit from a higher angular resolution, a higher imaging depth and/or may be located deeper within tissue, such that quality imaging is not possible without adjusting these and other parameters. Based on synchronization of the angular direction, the invention provides for dynamically adapting imaging parameters during the course of one rotation such that the image quality and/or the field of view is improved in a certain direction. Thus, improved imaging settings are possible for selected regions of interest while other regions around the circumference do not need these more demanding settings.

In non-limiting examples, systems of the invention accomplish selective rotational focusing by one or more of: changing the transmit/receive firing rate; changing the imaging depth; changing the planewave opening angle and steering angle, changing sampling rate on receive/transmit pulse shape and type, and transmit/receive sequence, and the like. The focus region is thus freely selected and adapted in the systems by adjusting the phase between motor speed and repeating firing patterns, and/or by dynamically adjusting the transmit/receive pattern. For example, the motor position and firing sequence may be modified to control the direction of the focus region. If a non-rotating ultrasound design is used, for example, a 2D matrix array system or any other form of 3D ultrasound system, firing and directional focusing, depth, and resolution may be controlled entirely through electronic element activation and delays (electronic beamforming).

Thus, systems and methods of the invention provide for controlling the direction of the focus region using various methods alone or in combination, including the firing rate and/or the rotational speed by mechanical and non-mechanical means to provide live 3D ultrasound imaging with an imaging quality that is selectively improved/adapted in specific directions of a region of interest.

In one aspect, the invention discloses a system for providing selective image focusing. The system includes a console configured to be operably associated with an ultrasound imaging device and to exchange data with the ultrasound imaging device. The console comprises, a hardware processor coupled to non-transitory, computer-readable memory containing instructions executable by the processor. The instructions cause the console to define a set of parameters associated with operation of an ultrasound transducer unit of an ultrasound imaging device to achieve an imaging characteristic of one or more images captured via the ultrasound imaging device for a first selected region of interest; and adjust the set of parameters. Adjusting the set of parameters provides directional focusing and/or imaging quality control at a second selected region of interest such that the imaging characteristic and/or an image quality is optimized for one or more directions at the second selected region of interest as compared to the first selected region of interest.

In some embodiments, the parameters comprise planewave or diverging wave front characteristics. Particularly, in some embodiments, the planewave or diverging wave characteristics comprise at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

In some embodiments, the imaging characteristic is associated with at least one of an angular resolution, a field of view, an imaging depth, a transmit/receive pattern, a transmit firing rate, and a planewave opening angle.

In various embodiments, the console is configured to dynamically define and/or adjust the set of parameters. For example, in some embodiments, the imaging characteristic is selected and adjusted by dynamically adapting the transmit/receive pattern in the first and/or second selected region of interest. Further, the first and/or second selected region of interest is defined based on one or more anatomical features. In some embodiments, the set of parameters is dynamically adjusted by optimizing imaging depth and imaging resolution to provide a focused view of the first and/or second selected region of interest. For example, in particular embodiments, the imaging resolution comprises one or more of depth, lateral resolution, and angular resolution.

In some embodiments, the system further comprises an ultrasound imaging device operably coupled to the console. The ultrasound imaging device may comprise an ultrasound transducer unit capable of full circumferential three-dimensional (3D) imaging.

The console, in some embodiments, further comprises a controller to enable capture of planewave or diverging wave acquisition data over a circumferential 360-degree imaging region, such that the first and/or second selected region of interest is within a circumference encompassing the 360-degree imaging region. In various embodiments, the controller is capable of controlling a rotary motor operably coupled to the ultrasound transducer unit to enable a continuous rotation or a positioning of the ultrasound transducer unit.

The console, in some embodiments, is configured to dynamically define and adjust the set of parameters during the course of one rotation such that the imaging characteristic and/or the image quality is optimized in a selected direction of the second selected region of interest as compared to the imaging characteristic and/or image quality within and/or outside the first selected region of interest. The console, in some embodiments is configured to dynamically define and adjust the set of parameters such that the imaging characteristic and/or the image quality is optimized over a continuous range of regions from the first selected region of interest to the second selected region of interest. The imaging characteristic and/or the image quality is optimized via linear interpolation of the set of parameters over the range of regions from the first selected region of interest to the second selected region of interest.

In some embodiments, the console is further configured to synchronize a firing rate and a firing direction of the ultrasound transducer unit. The firing direction may be defined through a rotational position of the array, or in the context of a 2D flat/flexible array, through electronic control of the respective transducer area. Further, the image characteristic in the second selected region of interest is adjusted by dynamically adjusting a phase between at least a motor speed and a firing rate of the ultrasound transducer unit, in some embodiments.

In some embodiments, the console is configured to adjust an imaging depth of the ultrasound transducer unit to thereby create an asymmetric imaging volume around the circumference to selectively perform imaging in a certain direction.

In other embodiments, the console is configured to adjust a firing rate of the ultrasound transducer unit to thereby achieve a higher angular resolution in the selected region of interest.

In particular embodiments, the console is configured to adjust a planewave or diverging wave front characteristics and a steering angle of the ultrasound transducer unit to thereby achieve a complex shape of an imaging volume in the selected region of interest.

In some embodiments, a rotational velocity of the ultrasound transducer unit is varied by a mechanical change in the ultrasound transducer unit. For example, in some embodiments, the mechanical change introduces friction inside the ultrasound transducer unit in one direction to slow rotation of the ultrasound transducer unit. In particular embodiments, the firing rate is varied to compensate for friction present inside the ultrasound transducer unit.

In other aspects, the invention discloses a method for providing selective image focusing. The method includes providing a console configured to be operably associated with an ultrasound imaging device and exchange data therewith; defining, via the console, a set of parameters associated with operation of an ultrasound transducer unit of the ultrasound imaging device to achieve an imaging characteristic of one or more images captured via the ultrasound imaging device for a first selected region of interest; and adjusting, via the console, the set of parameters, wherein adjusting the set of parameters provides directional focusing and/or imaging quality control at a second selected region of interest such that the characteristic and/or an image quality is optimized for one or more directions at the second selected region of interest as compared to the first selected region of interest.

In some embodiments of the method, the parameters comprise planewave or diverging wave front characteristics. In particular embodiments, the planewave or diverging wave characteristics comprise at least one of a virtual source focus, a distance, an opening angle, a steering angle, a number of individual firings, a transmit/receive pattern, a transmit firing rate, an imaging depth, a rotational velocity, a rotational position, and a 3-dimensional wave direction steering. Further, the imaging characteristic is associated with at least one of an angular resolution, a field of view, an imaging depth, a transmit/receive pattern, a transmit firing rate, and a planewave opening angle. In some embodiments of the methods, the console is configured to dynamically define and/or adjust the set of parameters. For example, in some embodiments, the imaging characteristic is selected and adjusted by dynamically adapting the transmit/receive pattern in the first and/or second selected region of interest. Further, in some embodiments of methods of the invention, the first and/or second selected region of interest is defined based on one or more anatomical features. The set of parameters is dynamically adjusted by optimizing imaging depth and imaging resolution to provide a focused view of the region of interest, in some embodiments of the methods. In particular embodiments, the imaging resolution comprises one or more of depth, lateral resolution, and angular resolution.

In various embodiments, the methods of the invention further include providing an ultrasound imaging device operably coupled to the console, the ultrasound imaging device comprising an ultrasound transducer unit capable of full circumferential three-dimensional (3D) imaging. In particular embodiments, the console further comprises a controller to enable capture of planewave or diverging wave acquisition data over a circumferential 360-degree imaging region, wherein the first and/or second selected region of interest is within a circumference encompassing the 360-degree imaging region. The controller is capable of controlling a bias voltage selectively applied to one or more of a plurality of first and/or second electrodes of a transducer comprising an array of individual imaging elements, wherein the bias voltage defines a voltage for a row or a column connected to the electrode to activate or deactivate imaging by the individual imaging elements in the row or column to define an angular imaging aperture.

In some embodiments of methods of the invention, the controller is capable of controlling a rotary motor operably coupled to the ultrasound transducer unit to enable a continuous rotation or a positioning of the ultrasound transducer unit. Further, the console is configured to dynamically define and adjust the set of parameters during the course of one rotation such that the imaging characteristic and/or the image quality is improved in a selected direction of the second selected region of interest as compared to the imaging characteristic and/or image quality outside the first selected region of interest, in some embodiments. In some embodiments, the console is configured to dynamically define and adjust the set of parameters such that the imaging characteristic and/or the image quality is optimized over a continuous range of regions from the first selected region of interest to the second selected region of interest. For example, in some embodiments, the imaging characteristic and/or the image quality is optimized via linear interpolation of the set of parameters over the range of regions from the first selected region of interest to the second selected region of interest.

In some embodiments of the methods of the invention, the console is further configured to synchronize a firing rate and a firing direction of the ultrasound transducer unit. The firing direction may be defined through a rotational position of the array, or in the context of a 2D flat/flexible array, through electronic control of the respective transducer area. In some embodiments, the image characteristic in the second selected region of interest is adjusted by dynamically adjusting a phase between at least a motor speed and a firing rate of the ultrasound transducer unit. In some embodiments, the console unit is configured to adjust an imaging depth of the ultrasound transducer unit to thereby create an asymmetric imaging volume around the circumference to selectively perform imaging in a certain direction. Further, the console unit is configured to adjust a firing rate of the ultrasound transducer unit to thereby achieve a higher angular resolution in the second selected region of interest, in some embodiments. The console unit is configured to adjust a planewave or diverging wave front characteristic and a steering angle of the ultrasound transducer unit to thereby achieve a complex shape of an imaging volume in the region of interest.

In some embodiments of methods of the invention, a rotational velocity of the ultrasound transducer unit is varied by a mechanical change in the ultrasound transducer unit. For example, in some embodiments, the mechanical change introduces friction inside the ultrasound transducer unit in one direction to slow rotation of the ultrasound transducer unit. In particular embodiments, the firing rate is varied to compensate for friction present inside the ultrasound transducer unit.

### BRIEF DESCRIPTION OF THE DRAWINGS

FIGS. 1A and 1B are diagrammatic illustrations of an exemplary ultrasound system for providing visualization and characterization of vasculature within a patient with which devices of the invention may be used.

FIG. 2 is a perspective view of an imaging catheter with which devices of the invention may be coupled.

FIG. 3 illustrates a system of the invention according to one embodiment of the invention.

FIG. 4 illustrates a diagram of the physical sensitivity of imaging elements radially around a cylindrical array catheter in some embodiments of a cylindrical transducer array used with systems of the invention.

FIG. 5 illustrates the active radial aperture for radial rows around a cylindrical imaging catheter used in some embodiments of the invention.

FIG. 6 illustrates changing the firing rate in a region of interest.

FIG. 7 illustrates adapting the imaging depth in a region of interest, according to one embodiment of the invention.

FIG. 8 illustrates changing the planewave opening angle and steering angle in a region of interest, according to one embodiment of the invention.

FIG. 9 further illustrates selective image focusing in a region of interest according to one embodiment of the invention.

FIG. 10 illustrates an embodiment of bias row activation in devices of the present invention.

FIG. 11 is a block diagram of a method for providing selective imaging focusing according to one embodiment of the invention.

### DETAILED DESCRIPTION

The present invention recognizes the drawbacks of current systems using an ultrasound probe for 3D imaging, namely the technical challenges that limit the imaging quality of specific anatomical regions of interest in the full 360-degree view. Imaging an anatomical region of interest may benefit from a higher angular resolution, a higher imaging depth and/or the anatomical region may be located deeper in tissue such that quality imaging is not possible without adjusting these and other parameters. In particular, current 2D and 3D ultrasound systems do not provide a means for selective directional focusing, depth adjustment, and/or resolution imaging, but instead apply one generalized beamforming and reconstruction approach to the entire volume at hand.

The invention recognizes that imaging specific anatomical structures that lay in a particular angular direction may be improved if imaging parameters can be selectively adjusted to vary the imaging quality. Accordingly, systems and methods of the invention provide for live ultrasound imaging using a rotating transducer array or a 3D ultrasound transducer to selectively improve or adapt the imaging quality in specific directions of a region of interest.

Specifically, the invention provides for selectively adjusting the imaging parameters to achieve, for example, a higher resolution, and/or deeper penetration into tissue, and/or improved imaging quality in an anatomical region of interest. Based on synchronization of the angular direction, the invention provides for dynamically adapting imaging parameters during the course of one rotation such that the imaging is optimized in a certain focus area. In non-limiting examples, systems of the invention accomplish selective rotational focusing by one or more of: changing the transmit/receive firing rate; changing the imaging depth, changing the planewave opening angle and steering angle; changing a sampling rate on receive/transmit pulse shape and type, and changing a transmit/receive sequence. Accordingly, image quality and/or the field of view is improved in the selected direction. As a result, optimized imaging settings are possible for selected regions of interest while other regions around the circumference do not need these more demanding settings.

By way of overview, and as is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). In an ultrasound exam, the imaging device, (i.e. the transducer, probe, or transducer probe) is placed directly on the skin or inside a body opening (e.g. endovascular ultrasound, intravascular ultrasound, intracardiac echocardiography). The final quality of the image obtained through ultrasound scanning is limited to the technical specifications of the equipment, the propagation of ultrasonic waves through the tissue analyzed, and the method used to reconstruct the images.

### Ultrasound Imaging System

Systems and methods of the invention address the technical challenges that limit the field of view and/or imaging quality of specific anatomical regions of interest in the full 360-degree view.

As described in more detail below, in some embodiments, the invention provides a system for selective imaging focusing to define a set of parameters associated with operation of an ultrasound transducer unit of an ultrasound imaging device to achieve an imaging characteristic of one or more images captured via the ultrasound imaging device for a first selected region of interest. The systems adjust the set of parameter to provide directional focusing and/or imaging quality control at a second selected region of interest such that the imaging characteristic and/or an image quality is optimized for one or more directions at the second selected region of interest as compared to the first selected region of interest.

Systems and devices of the invention may be manufactured and/or assembled using current approaches. Systems and devices of the method may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein in their entirety.

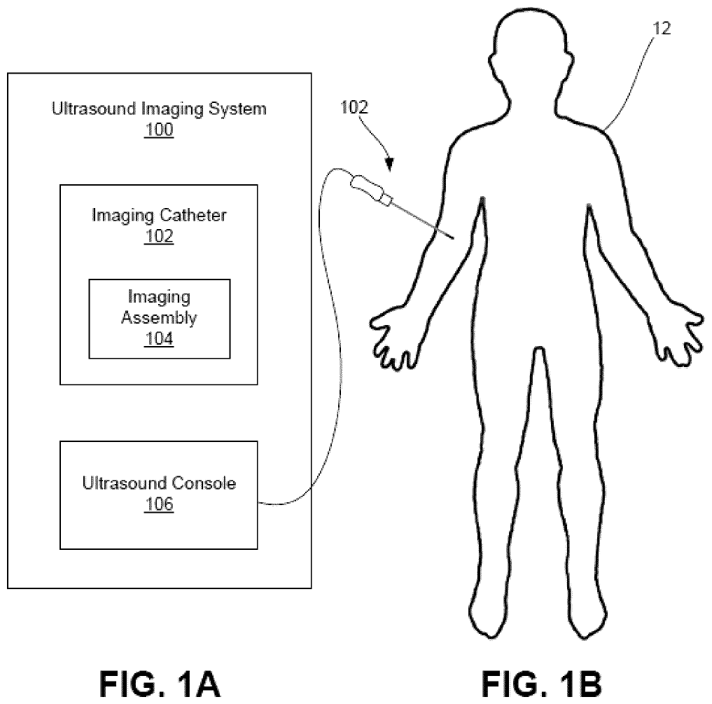

FIGS. 1A and 1B are diagrammatic illustrations of an exemplary ultrasound system/device 100 for providing visualization and characterization of, for example, vasculature within a patient 12 with which systems and methods of the invention may be used. Generally, the ultrasound system/device 100 includes an imaging catheter 102 equipped with an imaging assembly 104 and an ultrasound console 106 to which the imaging catheter 102 is to be connected.

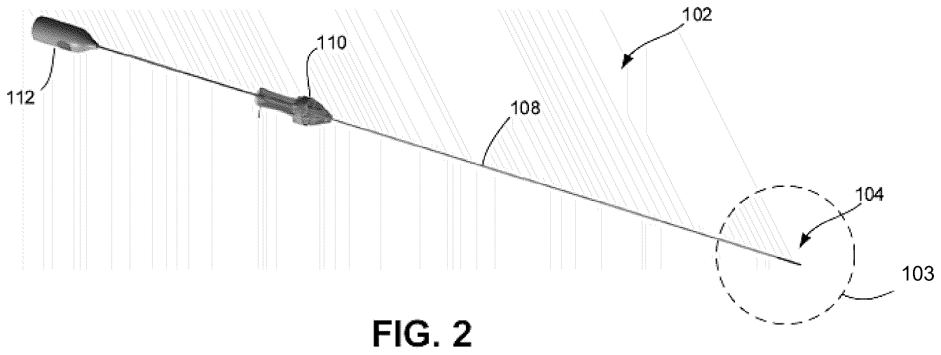

FIG. 2 is a perspective view of an imaging catheter 102 with which the systems may be used. The catheter 102 may include a catheter body 108, including proximal and distal portions. The imaging assembly 104 may be provided at the distal portion, for example, generally defining a distal end of an imaging catheter 103. A handle 110 may be operably associated with the catheter body 108 and allow for an operator (i.e., surgeon or other medical professional) to manipulate and advance the imaging assembly 104 and the catheter body 108 to a desired target site within the patient's vasculature. The handle 110 may include user-operable inputs for controlling various features and functions of the imaging assembly 104. An interface member 112 may be provided at a distal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

### Selective Image Focusing

Circumferential 3D ultrasound imaging via a rotating transducer or 2D array electronic scanning, allows for obtaining a rotationally symmetric imaging volume. However, the maximum transmit/receive firing rate is constrained by the electronic design. As a result, because of processing constraints, current circumferential 3D ultrasound imaging uses a constant rotational velocity as well as a constant firing rate, resulting in a constant angular resolution around the circumference.

For example, in circumferential 3D ultrasound imaging via a rotating transducer, and also in 2D matrix array systems, the maximum transmit/receive firing rate is constrained by the electronic design, which, specifically for matrix array transducers, often requires micro-beamforming steps directly in the transducer in order to reduce data rates to the system. The maximum firing rate is also limited by the penetration depth. For example, due to the speed of sound being 1540 m/s on average, a roundtrip to a depth of 40 mm takes 50 microseconds (20 kHz firing). A roundtrip to a depth of 100 mm takes about 130 microseconds (7.6 kHz firing). Requiring the deepest penetration depth throughout the whole volume significantly limits the firing rate.

Further, resulting data rates are dependent on the Receive Analog to Digital Conversion (A2D), where deeper penetration results in more samples required for each firing channel (e.g. 40 megasamples per second with a sampled time of 50 microseconds for 40 mm depth, and 130 microseconds for 100 mm depth resulting in 2000 samples for 40 mm, and 4000 samples for 100 mm). This requires much higher data rates to be transmitted throughout the imaging and processing steps. For applications where not all processing happens directly linked to A2D conversion (e.g. distributed systems, catheter applications, software-defined imaging pipelines), this poses strong challenges on system design for sustainable processing and transmission data rates.

These considerations are limiting factors in current 3D and 4D imaging systems with matrix array probes and cylindrical imaging systems. As a result, and in contrast to the present invention, current 2D and 3D ultrasound systems do not provide a means for directional focusing optimized for a certain area, but instead apply one generalized beamforming and reconstruction approach to the entire volume at hand.

The systems and methods disclosed herein allow for dynamically adapting relevant ultrasound imaging parameters to provide optimized imaging in a selected region of interest. Improved imaging may be, for example, improved resolution and/or depth where needed. Thus, the invention avoids the limitations—i.e. much higher data rates, longer firing sequences, and resource overhead for individual element control-associated with applying a homogeneous pattern for the entire volume.

The invention recognizes that specific anatomical structures may lay in a particular angular direction that would benefit from a higher resolution and/or higher imaging depth, are located deeper in tissue, or would otherwise require improved imaging settings, while other regions around the circumference do not require such demanding settings. The invention provides for improved angular resolution in a specific region of interest by selectively varying, adjusting, and/or adapting acquisition resources, i.e. parameters and settings. Because acquisition resources are limited, the invention provides for selectively varying these resources to focus on important structures of interest. The systems and methods disclosed herein selectively improve or adapt the imaging parameters in specific directions of a region of interest to provide optimized and/or enhanced, live ultrasound imaging.

In one aspect, the invention discloses a system for selective imaging focusing.

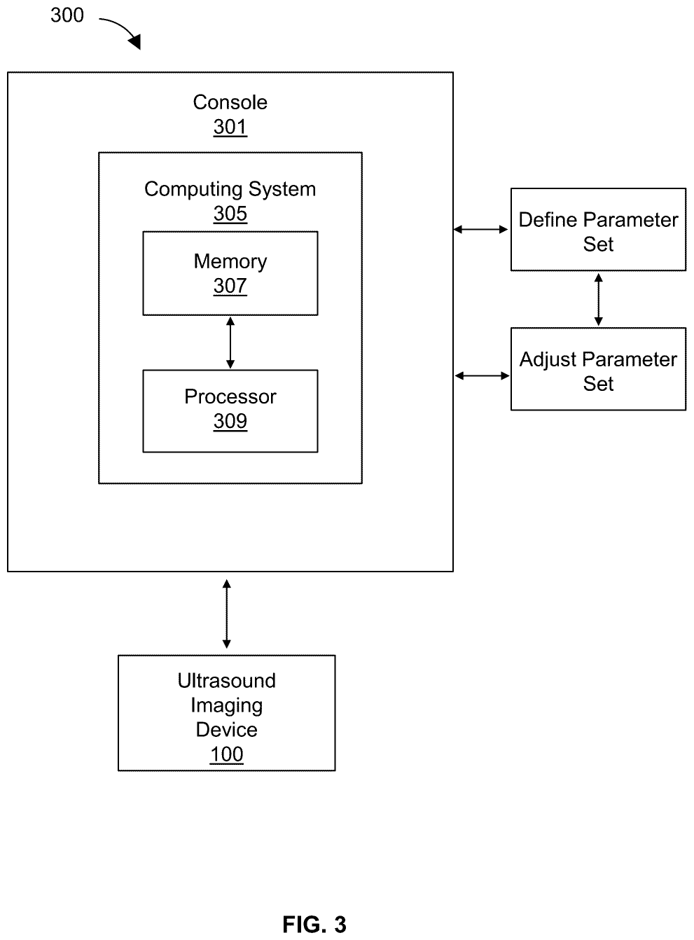

FIG. 3 illustrates a system 300 of the invention according to one embodiment of the invention. The systems include a console 301 configured to be operably associated with an ultrasound imaging device 100 and exchange data therewith. The console may include a hardware processor 309 coupled to non-transitory, computer-readable memory 307. The non-transitory, computer-readable memory 307 may include instructions executable by the processor to cause the console to define and adjust parameters for ultrasound imaging in a selected region of interest. The console may include a computing system 305 such that the computing system, via the memory, causes the console to define and adjust a set of imaging parameters for a selected region of interest.

As described in detail herein, the console defines a set of parameters associated with operation of an ultrasound transducer unit of an ultrasound imaging device to achieve an imaging characteristic of one or more images captured via the ultrasound imaging device for a first selected region of interest. The console then adjusts the set of parameters. Adjusting the parameters provides directional focusing and/or imaging quality control at a second selected region of interest such that the imaging characteristic and/or an image quality is optimized for one or more directions at the second selected region of interest as compared to the first selected region of interest.

Accordingly, a set of parameters may be defined for the volume as a whole and then adjusted to optimize imaging in a selected region of interest or focus region within the volume. The systems allow for defining multiple regions of interest by selectively adapting imaging parameters to achieve one or more desired imaging characteristics in the region of interest. The systems may define a region of interest as the image as a whole and also define a region or regions of interest within the full image to selectively optimize imaging within the defined region(s). This allows for having multiple regions of interest defined, for example with one region being the full image and one or more others being a certain focus region. Thus, systems and methods of the invention provide for optimized imaging, for example, improved imaging contrast, resolution, sensitivity, and/or depth, in the focus region as compared to the volume as a whole.

The systems defines the set of parameters associated with operation of an ultrasound transducer unit of an ultrasound imaging device to achieve an imaging characteristic of one or more images captured via the ultrasound imaging device. The imaging device may be, for example, a rotating transducer array or a 3D ultrasound transducer. Thus, the concepts may be applied to 3D circumferential rotating imaging as well as 2D matrix array imaging systems where both the penetration depth as well as (spatial) resolution may be specifically optimized to provide a focused view of the desired target region/anatomy of interest, while the full volumetric information is available for providing enhanced anatomical context to the user.

In some embodiments, the parameters comprise planewave or diverging wave front characteristics. For example, the planewave or diverging wave characteristics may be, in non-limiting examples, at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

Ultrafast ultrasound imaging techniques, such as planewave or diverging wave imaging, may be required to enable imaging within the constraints of the application, particularly for intravascular and/or intracardiac tissue imaging. Systems and methods of the invention allow for the direct utilization of all native ultrafast imaging techniques. For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

In the present invention, the transducer may be any type for transmitting and receiving acoustic waves. For example, the transducer may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices do not require bias for achieving imaging sensitivity.

The transducer may be a micro-electromechanical systems (MEMS)-based capacitive micromachined ultrasonic transducer (CMUT) configured as a two-dimensional (2D) array structure. In non-limiting examples, the 2D array may be a flexible structure. The cylindrical imaging array may consist of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13 (10) 3873-3882, incorporated herein by reference.

Additionally and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers may be made of electrostrictive materials such as an electrostrictive polymer, or any material that can be activated using bias voltage to achieve imaging sensitivity.

As noted, systems of the invention define a set of parameters associated with operation an ultrasound transducer unit of an ultrasound imaging device. The parameters may be associated with planewave or diverging wave characteristics. As described in more detail herein, the planewave or diverging wave characteristics may be, for example, a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

In this context, virtual source focus include methods that coherently combine the recorded data from multiple transmissions to form a synthetic focus making geometric assumptions about the transmissions. These also include diverging waves (virtual source behind the array) and plane waves (virtual source at infinity). Further, the steering angle is not limited to certain directions, but rather encompasses both lateral and elevational steering angle. To avoid bias by the word “angle” the steering angle is understood to mean steering in 3D.

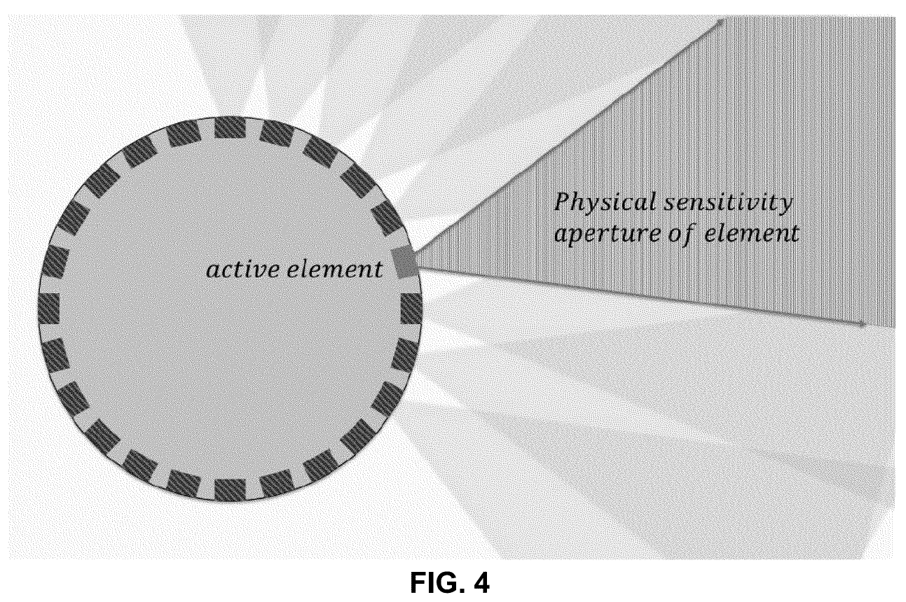

FIG. 4 illustrates a diagram of the physical sensitivity of imaging elements radially around a cylindrical array catheter in some embodiments of a cylindrical transducer array. The cylindrical shape (or radius of the cylinder) provides geometrical boundary constraints to which elements can receive data for a point in space. For an active element, the physical sensitivity aperture of the element is illustrated. Sensitivity is an important parameter to describe the electro-transducer energy conversion efficiency and is a key indicator of transducer performance. Generally, sensitivity is defined as the ratio of an output quantity to an input quantity.

The aperture is the active area that transmits or receives acoustic wave at certain moment. For a single-element transducer, the aperture size is the transducer element size. For a transducer with an array of elements, the aperture is all the elements that are active simultaneously. As is generally understood, ultrasound imaging has a spatially variant resolution that depends on the size of the active aperture of the transducer (including the dimensions of each ultrasound element), the center frequency and bandwidth of the transducer, and the selected transmit pattern. For focused imaging, lateral resolution is best at the focal length distance and widens away from this distance in a nonuniform way because of diffraction effects caused by apertures on the order of a few to tens of wavelengths. For non-focused imaging such as planewave or diverging wave imaging, lateral resolution for a transmit widens away from the transducer surface, as focusing is performed using multiple transmit waves.

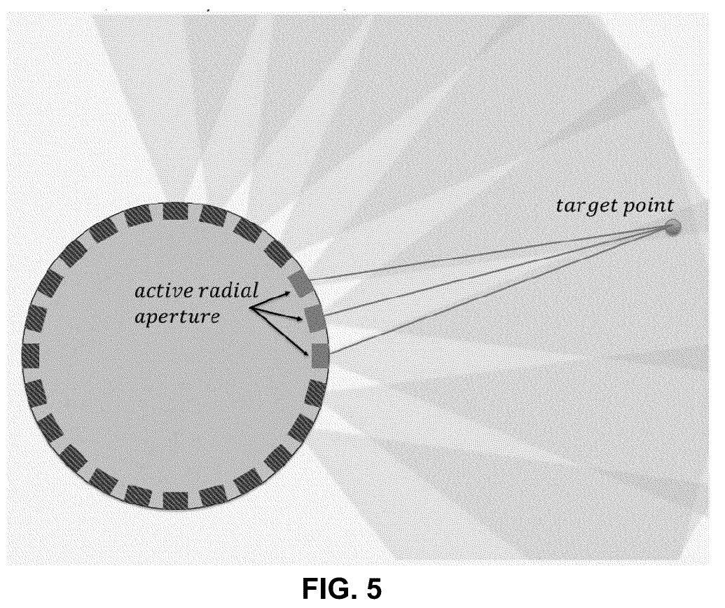

FIG. 5 illustrates the active radial aperture for radial rows around a cylindrical imaging catheter used in some embodiments of the invention. The active aperture depends on the angular coverage of each physical element (i.e. the directivity of imaging elements). Each ultrasound transducer has its own specific directivity pattern. The directivity pattern, also known as beam pattern or radiation pattern, is an important far-field characteristic of a transducer. Directivity pattern consists of a main lobe and side lobes. Radiation intensity is dominant mainly in the front region of the transducer source, so the main lobe is directly in front of the ultrasound transmitter, followed by side lobes sidewise with null region in between these lobes. In general, the directivity patterns are the same whether the transducer is used as a transmitter or as a receiver. Systems of the invention may use a flexible imaging array system to allow for flexibility in convex aperture selection and optimization, for example, one capable of row-column addressing in combination with bias activation.

The flexible imaging arrays for ultrafast ultrasound imaging may be configured for any shape, for example cylindrical/non-cylindrical, flat and non-flat. The flexible arrays may allow for defining and optimizing an angular aperture for any shaped surface but particularly for a concave surface, e.g. a cylindrical array.

It should be noted that specific descriptions of the present invention include using the systems of the invention for ultrasound visualization of intravascular and/or intracardiac tissue, which may be particularly useful for catheter-based interventional procedures for assessing the anatomy as well as functional data in relation to the target volume of interest. However, as is generally understood, the systems and methods of the present invention may be used for ultrasound visualization of tissue of any kind with respect to any kind of procedure in which imaging analysis is used and/or preferred.

In some embodiments, the imaging characteristic is associated with at least one of an angular resolution, a field of view, an imaging depth, a transmit/receive pattern, a transmit firing rate, and a planewave opening angle.

For example, the systems may first synchronize a current angular direction and then dynamically adapt other imaging parameters to achieve improved imaging. Based on the synchronization of angular direction, the imaging parameters may be dynamically adapted during the course of one rotation/volume such that the image quality and/or field of view is improved in a certain direction, such that the imaging achieves a desired optimization.

As disclosed herein, the console may be configured to dynamically define and/or adjust the set of parameters. Thus, the parameters may be adjusted during the course of image capture, for example during the course of one or more rotations.

The selective image focusing may be achieved in a number of different ways. For example, by first achieving synchronization between the firing and the rotational position. Then, selectively improving image quality or field of view in a certain direction may be achieved by configuring specific ultrasound firing patterns. The phase between the motor position and the firing sequence may be modified to control the direction of the “focus region”. If a non-rotating ultrasound design is used (e.g. in a 2D-matrix array system or any other form of 3D ultrasound system), firing and directional focusing/depth/resolution can be controlled entirely through electronic element activation and delays (electronic beamforming).

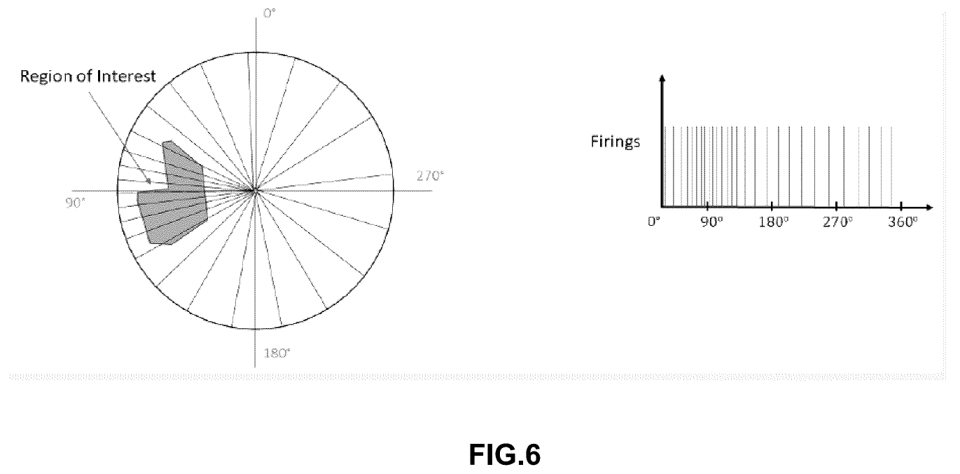

FIG. 6 illustrates changing the firing rate in a region of interest, for example by applying a higher firing rate in the region of interest. A higher firing rate allows for more beams and thus higher resolution in the target tissue. In some embodiments, the imaging characteristic is selected and adjusted by dynamically adapting the transmit/receive pattern in the first and/or second selected region of interest. As an example, the transmit/receive firing rate may be adapted or changed to achieve a higher angular resolution in a selected directions. Specifically, the firing rate may be increased in a selected region of interest during the course of the rotation to achieve a higher angular resolution in the region of interest. Thus firing rate may be associated with the density of firings in a certain region. As such the firing rate may be of a higher density as compared to the density of firings in the rest of the volume of the rotation.

Imaging systems are fundamentally limited by the ballistic time of ultrasound waves to reach/reflect in tissue, e.g. 1540 m/s in soft tissue. Systems are limited to transmit/receive on only part of the full 2D array, or, if using a rotational array concept, the transmit/receive area is limited by the rotation of the array. Thus to maximize firing density in one region, the overall volumetric imaging rate, i.e. the imaging rate of the whole rotation, may need to be lowered (e.g. instead of 20 Hz imaging at 10 Hz only), or the firing density or penetration depth may be lowered in a certain region. Thus, the systems allow for selectively changing these parameters as they apply to imaging the whole rotation as well as selectively optimizing imaging within the volume in a region of interest.

As disclosed herein, the systems provides for continuous adaptation of imaging parameters. For example, while there may be one or more distinct imaging parameters or qualities defined by the system for inside and outside a region of interest, the systems also allows for a continuous blending of parameters instead of discrete parameter sets for distinct regions. Thus, the systems allows for changing from discrete parameter definitions in regions of interest to continuous adaptation of parameters to provide for continuous blending of imaging.

As discussed in detail above, the systems may comprise an ultrasound imaging device operably coupled to the console. The ultrasound imaging device may include an ultrasound transducer unit capable of full circumferential three-dimensional (3D) imaging. In some embodiments, the console further comprises a controller to enable capture of planewave or diverging wave acquisition data over a circumferential 360-degree imaging region, wherein the first and/or second selected region of interest is within a circumference encompassing the 360-degree imaging region.

Further, the systems may use bias voltage to tune the frequency range of the imaging array within a region of interest, with the sensitivity for a frequency range being driven by the bias voltage. Bias voltage may be used to deactivate imaging elements entirely in the region of interest or within the volume as a whole. Importantly, using bias voltage to activate imaging elements (i.e. cells) allows for imaging without multiplexing. Bias activation means that when a nominal bias voltage is applied to tune the imaging array to a target center frequency imaging will be activated on this row or column. Similarly, applying a bias voltage of OV or a voltage level where the sensitivity of the imaging element is minimal deactivates the imaging row or column. This allows for the enablement or disablement of full rows or columns on the array and provides for employing a flexible transmit/receive scheme on the activated row(s) and/or column(s) for electronic focusing.

In some embodiments, the controller is capable of controlling a rotary motor operably coupled to the ultrasound transducer unit to enable a continuous rotation or a positioning of the ultrasound transducer unit.

In some embodiments, the console may be configured to dynamically define and adjust the set of parameters during the course of one rotation such that the imaging characteristic and/or the image quality is optimized in a selected direction of the second selected region of interest as compared to the imaging characteristic and/or image quality within and/or outside the first selected region of interest.

Further, in some embodiments, the console is configured to dynamically define and adjust the set of parameters such that the imaging characteristic and/or the image quality is optimized over a continuous range of regions from the first selected region of interest to the second selected region of interest. The imaging characteristic and/or the image quality may be optimized, for example, via linear interpolation of the set of parameters over the range of regions from the first selected region of interest to the second selected region of interest.

As noted above, in some embodiments, console is further configured to synchronize a firing rate and a firing direction of the ultrasound transducer unit. The firing direction may be defined through a rotational position of the array, or in the context of a 2D flat/flexible array, through electronic control of the respective transducer area. The image characteristic in the second selected region of interest may be adjusted by dynamically adjusting a phase between at least a motor speed and a firing rate of the ultrasound transducer unit.

In some embodiments, the set of parameters may be dynamically adjusted by optimizing imaging depth and imaging resolution to provide a focused view of the first and/or second selected region of interest.

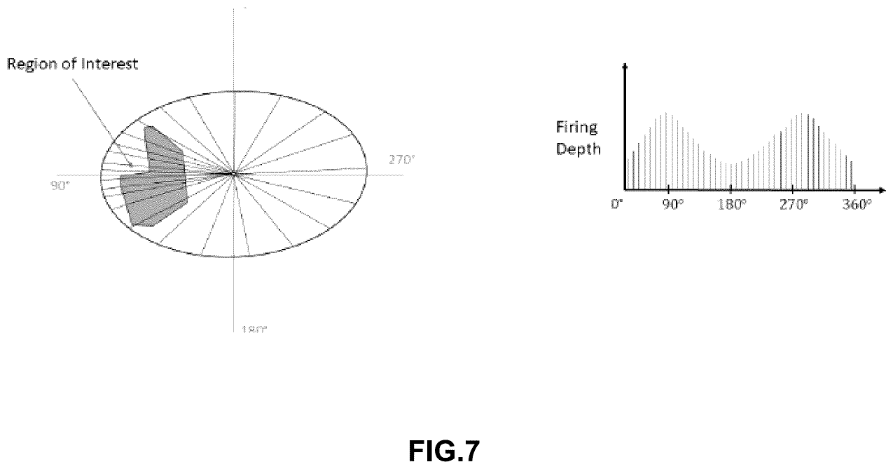

FIG. 7 illustrates adapting the imaging depth in a region of interest, according to one embodiment of the invention. Changing the imaging depth may enable the creation of asymmetric imaging volumes around the circumference to selectively perform imaging of deeper structures in certain directions. The imaging depth may be kept constant in other directions where deeper tissue imaging is not important. This concept may be applied to electronic focusing/multiplexing as well as mechanical rotation. In some embodiments the imaging resolution includes one or more of depth, lateral resolution, and angular resolution.

In some embodiments of the systems of the invention, the console is configured to adjust an imaging depth of the ultrasound transducer unit to thereby create an asymmetric imaging volume around the circumference to selectively perform imaging in a certain direction. As disclosed herein, the console may be configured to adjust a firing rate of the ultrasound transducer unit to thereby achieve a higher angular resolution in the selected region of interest.

Further, the console may be configured to adjust a planewave or diverging wave front characteristics and a steering angle of the ultrasound transducer unit to thereby achieve a complex shape of an imaging volume in the selected region of interest.

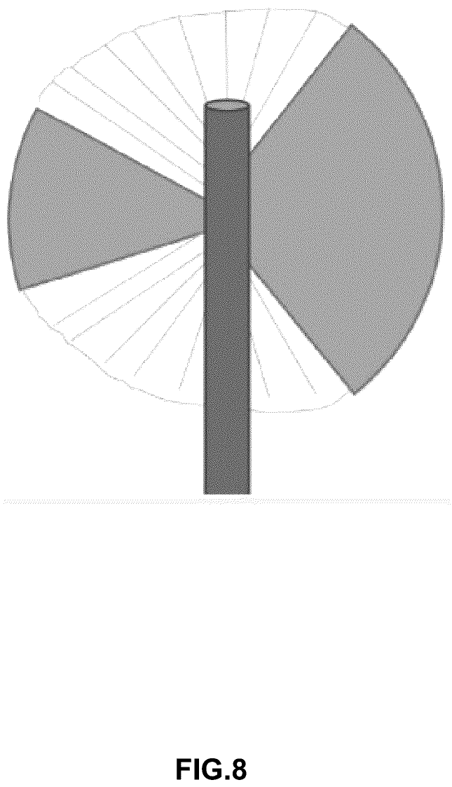

FIG. 8 illustrates changing the planewave opening angle and steering angle in a region of interest, according to one embodiment of the invention. Changing the planewave opening angle and steering angle yields complex shapes of the imaging volume that may be adapted to image a certain volume of interest. For example, in one direction, the planewaves may extend in a wide angle to achieve a wide opening angle in that direction while also having a narrow configuration in the other direction in order to achieve a higher imaging quality in that direction. This may be useful in intracardiac imaging when an intracardiac imaging catheter is placed in the right atrium to selectively provide deeper imaging with higher resolution in the septal region. For more lateral regions of this placement, for example the lungs, deeper penetration would not be required for anatomical context.

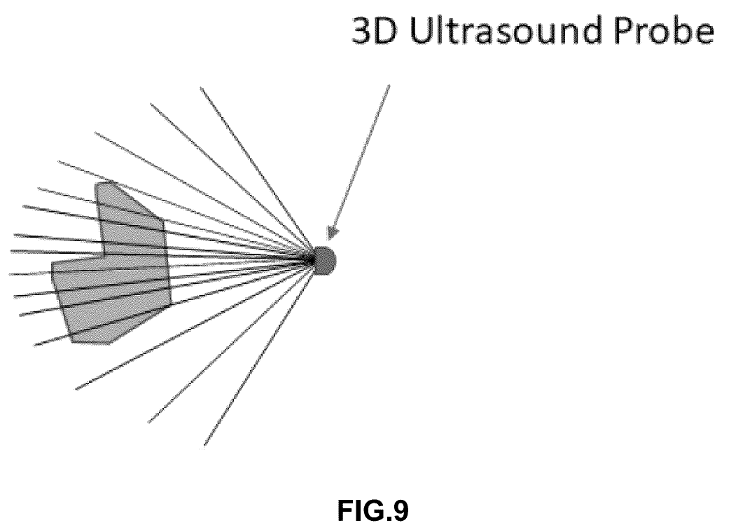

FIG. 9 further illustrates selective image focusing in a region of interest according to one embodiment of the invention. When good synchronization between the firing and the rotational position is achieved, selective image focusing may be achieved by configuring specific ultrasound firing patterns. The phase between the motor position and the firing sequence may be modified to control the direction of the “focus region”. If a non-rotating ultrasound design is used (e.g. in a 2D-matrix array system or any other form of 3D ultrasound system), firing and directional focusing/depth/resolution may be controlled entirely through electronic element activation and delays (electronic beamforming).

In some embodiments, the invention provides mechanical solution for non-uniform rotation speed and firing rate. For example, instead of varying the firing rate, rotational speed may be varied by a mechanical change in the catheter to introduce friction inside the catheter in one direction. In some embodiments, a rotational velocity of the ultrasound transducer unit is varied by a mechanical change in the ultrasound transducer unit.

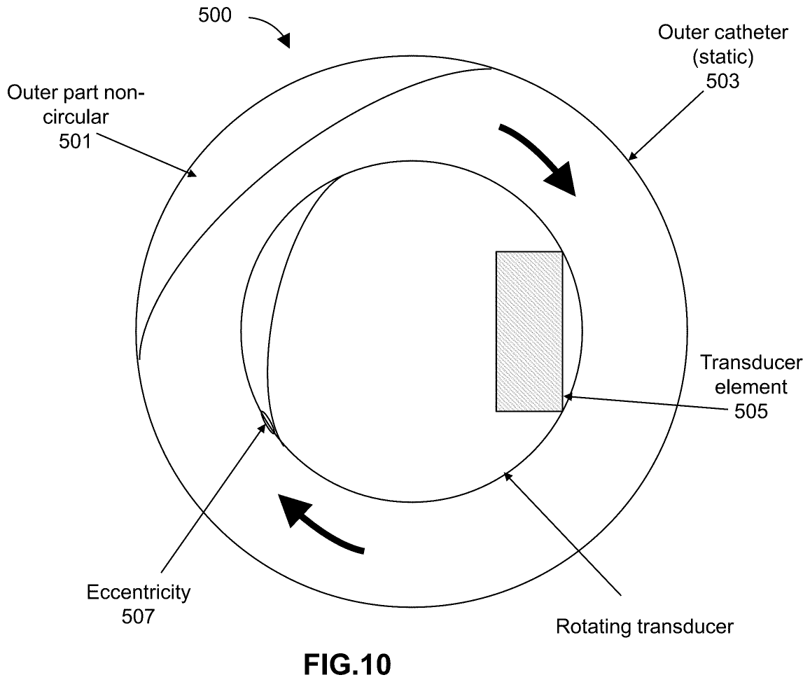

FIG. 10 illustrates one embodiment of a rotating transducer 500 with a non-circular outer portion 501 and a static outer catheter 503. For a rotating transducer with a constant firing rate, slowing down the rotation of the transducer using the mechanical change results in more firings from the transducer elements 505 in the region in which the rotation is slowed. With a constant firing rate, this will result in more firings in the direction. For example, the mechanical change may be an area of eccentricity 507 in the rotating transducer. The eccentricity may be a result of, for example, rubber or other material added to produce friction in the rotation of the transducer.

In some embodiments, the mechanical change introduces friction inside the ultrasound transducer unit in one direction to slow rotation of the ultrasound transducer unit. However, the systems also allows for compensating for mechanical behavior if friction is present inside the transducer unit such that it rotates slower in certain regions. Varying the firing rate in regions of interest or the volume as a whole compensates for this mechanical behavior. Thus, in some embodiments, the firing rate is varied to compensate for friction present inside the ultrasound transducer unit. In some embodiments, the motor is non-discrete in order to change from one set of parameters to the next. In other embodiments of the system, the motor is a discrete function.

As noted above, the selected region of interest may be based on certain anatomical features. In some embodiments, the first and/or second selected region of interest is defined based on one or more anatomical features, for example a specific ablation path. Thus, the selected region of interest or focused region may be defined based on the anatomy imaged.

The systems and methods of the invention provide for software-controlled adaptation of imaging parameters, for example, through software controlled focus-region steering. In any of the variations, the focus region may be freely chosen and adapted in software by adapting the phase between motor speed and repeating firing patterns, or by adapting the transmit/receive pattern. Adaptation may be performed dynamically during imaging.

The direction of the focus region may be controlled several ways. For example, the focus region may be directly exposed to the user via a user interface element, for example a direct control of the angle of the focus region, or by setting the angle of the focus region via indicating a specific region of interest in the image directly with the use of a mouse. The focus region may be implicitly controlled and/or adapted in combination with a 3D visualization of the data. In this way, the focus region will be implicitly tied to the region that is in the central focus of the 3D visualization. If the viewpoint of the 3D visualization changes, the imaging focus region is automatically adapted. This also takes into account knowledge about regions that are not displayed at all (outside of the current visualization field of view, or cropped away with a digital cropping function). The focus region may be automatically controlled by semantic analysis of the imaging data, to automatically detect particular anatomical regions and focus on these without user input, or by suggesting these regions to the user to focus on with a single interaction.

The console may include a computer program comprising an algorithm for evaluating, calculating, and optimizing the imaging parameters, as well as reconstructing the images using non-homogeneous spatial resolution and/or depth. As disclosed herein the imaging parameters may be, in non-limiting examples, planewave or diverging wave front characteristics. The planewave or diverging wave characteristics may be at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering. The imaging characteristics may be associated with, in non-limiting examples, at least one of an angular resolution, a field of view, an imaging depth, a transmit/receive pattern, a transmit firing rate, and a planewave opening angle.

Using defined algorithms, the console may be configured to provide feedback to an operator before or during operation of the imaging array for evaluating, calculating, and optimizing the imaging parameters. Thus, the systems provides for visualization of the reconstructed information assuming non-homogeneous spatial resolution or depth.

In some embodiments of the system, the console is in active communication with a computing system comprising an algorithm for evaluating, calculating, and selectively optimizing the imaging parameters.

As previously noted, the transducer probe may be operably coupled to the console, which may generally control operation of the transducer probe (i.e., transmission of sound waves from the probe) and/or the controller. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

For example, in an exemplary embodiment, the console may generally include a computing device configured to communicate across a network. The computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for optimizing imaging parameters and subsequent reconstruction into 2D and/or 3D images of the non-homogenous data.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. The console may further provide control over an imaging assembly, including control over the emission of ultrasound pulses therefrom (intensity, frequency, duration, etc.) as well as control over the movement of the ultrasound transducer unit.

### Method for Selective Image Focusing

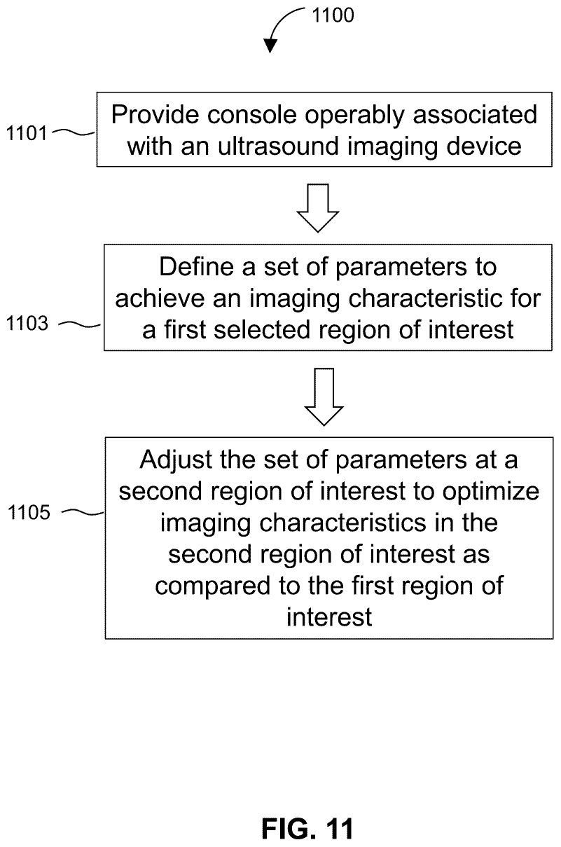

FIG. 11 illustrates an embodiment of a method 1100 for selective image focusing according to one embodiment of the invention. The method includes the steps of providing 1101 a console configured to be operably associated with an ultrasound imaging device and exchange data therewith; defining 1103 defining, via the console, a set of parameters associated with operation of an ultrasound transducer unit of the ultrasound imaging device to achieve an imaging characteristic of one or more images captured via the ultrasound imaging device for a first selected region of interest; and adjusting 1105, via the console, the set of parameters, wherein adjusting the set of parameters provides directional focusing and/or imaging quality control at a second selected region of interest such that the characteristic and/or an image quality is optimized for one or more directions at the second selected region of interest as compared to the first selected region of interest.

As described in detail herein, the console defines a set of parameters associated with operation of an ultrasound transducer unit of an ultrasound imaging device to achieve an imaging characteristic of one or more images captured via the ultrasound imaging device for a first selected region of interest. The console is configured to adjust the set of parameters. Adjusting the parameters provides directional focusing and/or imaging quality control at a second selected region of interest such that the imaging characteristic and/or an image quality is optimized for one or more directions at the second selected region of interest as compared to the first selected region of interest.

Accordingly, a set of parameters may be defined for the volume as a whole and then adjusted to optimize imaging in a selected region of interest or focus region within the volume. The methods allow for defining multiple regions of interest by selectively adapting imaging parameters to achieve one or more desired imaging characteristics in the region of interest. The methods may define a region of interest as the image as a whole and also define a region or regions of interest within the full image to selectively optimize imaging within the defined region(s). This allows for having multiple regions of interest defined, for example with one region being the full image and one or more others being a certain focus region. Thus, the methods of the invention provide for optimized imaging, for example, improved imaging contrast, resolution, sensitivity, and/or depth, in the focus region as compared to the volume as a whole.

The methods define the set of parameters associated with operation of an ultrasound transducer unit of an ultrasound imaging device to achieve an imaging characteristic of one or more images captured via the ultrasound imaging device. The imaging device may be, for example, a rotating transducer array or a 3D ultrasound transducer. Thus, the concepts may be applied to 3D circumferential rotating imaging as well as 2D matrix array imaging systems where both the penetration depth as well as (spatial) resolution may be specifically optimized to provide a focused view of the desired target region/anatomy of interest, while the full volumetric information is available for providing enhanced anatomical context to the user.

In some embodiments of the methods, the parameters comprise planewave or diverging wave front characteristics. For example, the planewave or diverging wave characteristics may be, in non-limiting examples, at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

Ultrafast ultrasound imaging techniques, such as planewave or diverging wave imaging, may be required to enable imaging within the constraints of the application, particularly for intravascular and/or intracardiac tissue imaging. Methods of the invention allow for the direct utilization of all native ultrafast imaging techniques. For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

As noted, in the present invention, the transducer may be any type for transmitting and receiving acoustic waves. For example, the transducer may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices do not require bias for achieving imaging sensitivity.

The transducer may be a micro-electromechanical systems (MEMS)-based capacitive micromachined ultrasonic transducer (CMUT) configured as a two-dimensional (2D) array structure. In non-limiting examples, the 2D array may be a flexible structure. The cylindrical imaging array may consist of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13 (10) 3873-3882, incorporated herein by reference.

Additionally and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers may be made of electrostrictive materials such as an electrostrictive polymer, or any material that can be activated using bias voltage to achieve imaging sensitivity.

Methods of the invention define a set of parameters associated with operation an ultrasound transducer unit of an ultrasound imaging device. The parameters may be associated with planewave or diverging wave characteristics. As described in more detail herein, the planewave or diverging wave characteristics may be, for example, a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

In this context, virtual source focus include methods that coherently combine the recorded data from multiple transmissions to form a synthetic focus making geometric assumptions about the transmissions. These also include diverging waves (virtual source behind the array) and plane waves (virtual source at infinity). Further, the steering angle is not limited to certain directions, but rather encompasses both lateral and elevational steering angle. To avoid bias by the word “angle” the steering angle is understood to mean steering in 3D.

It should be noted that specific descriptions of the present invention include using the methods of the invention for ultrasound visualization of intravascular and/or intracardiac tissue, which may be particularly useful for catheter-based interventional procedures for assessing the anatomy as well as functional data in relation to the target volume of interest. However, as is generally understood, the methods of the present invention may be used for ultrasound visualization of tissue of any kind with respect to any kind of procedure in which imaging analysis is used and/or preferred.

In some embodiments of the method, the imaging characteristic is associated with at least one of an angular resolution, a field of view, an imaging depth, a transmit/receive pattern, a transmit firing rate, and a planewave opening angle.

For example, the method may first synchronize a current angular direction and then dynamically adapt other imaging parameters to achieve improved imaging. Based on the synchronization of angular direction, the imaging parameters may be dynamically adapted during the course of one rotation/volume such that the image quality and/or field of view is improved in a certain direction, such that the imaging achieves a desired optimization.

As disclosed herein, the console may be configured to dynamically define and/or adjust the set of parameters. Thus, the parameters may be adjusted during the course of image capture, for example during the course of one or more rotations.

As disclosed herein, the selective image focusing may be achieved in a number of different ways. For example, by first achieving synchronization between the firing and the rotational position. Then, selectively improving image quality or field of view in a certain direction may be achieved by configuring specific ultrasound firing patterns. The phase between the motor position and the firing sequence may be modified to control the direction of the “focus region”. If a non-rotating ultrasound design is used (e.g. in a 2D-matrix array system or any other form of 3D ultrasound system), firing and directional focusing/depth/resolution can be controlled entirely through electronic element activation and delays (electronic beamforming).

In some embodiments of the method, the imaging characteristic is selected and adjusted by dynamically adapting the transmit/receive pattern in the first and/or second selected region of interest. As an example, the transmit/receive firing rate may be adapted or changed to achieve a higher angular resolution in a selected directions. Specifically, the firing rate may be increased in a selected region of interest during the course of the rotation to achieve a higher angular resolution in the region of interest. Thus firing rate may be associated with the density of firings in a certain region. As such the firing rate may be of a higher density as compared to the density of firings in the rest of the volume of the rotation.

Imaging systems are fundamentally limited by the ballistic time of ultrasound waves to reach/reflect in tissue, e.g. 1540 m/s in soft tissue. Systems are limited to transmit/receive on only part of the full 2D array, or, if using a rotational array concept, the transmit/receive area is limited by the rotation of the array. Thus to maximize firing density in one region, the overall volumetric imaging rate, i.e. the imaging rate of the whole rotation, may need to be lowered (e.g. instead of 20 Hz imaging at 10 Hz only), or the firing density or penetration depth may be lowered in a certain region. Thus, the methods allow for selectively changing these parameters as they apply to imaging the whole rotation as well as selectively optimizing imaging within the volume in a region of interest.

As disclosed herein, the methods of the invention provide for continuous adaptation of imaging parameters. For example, while there may be one or more distinct imaging parameters or qualities defined by the system for inside and outside a region of interest, the systems also allows for a continuous blending of parameters instead of discrete parameter sets for distinct regions. Thus, the methods allow for changing from discrete parameter definitions in regions of interest to continuous adaptation of parameters to provide for continuous blending of imaging.

As discussed in detail above, the methods may comprise an ultrasound imaging device operably coupled to the console. The ultrasound imaging device may include an ultrasound transducer unit capable of full circumferential three-dimensional (3D) imaging. In some embodiments, the console further comprises a controller to enable capture of planewave or diverging wave acquisition data over a circumferential 360-degree imaging region, wherein the first and/or second selected region of interest is within a circumference encompassing the 360-degree imaging region.

As previously disclosed, in some embodiments of the method, the controller is capable of controlling a bias voltage selectively applied to one or more of a plurality of first and/or second electrodes of a transducer comprising an array of individual imaging elements. Thus, the bias voltage defines a voltage for a row or a column connected to the electrode to activate or deactivate imaging by the individual imaging elements in the row or column to define an angular imaging aperture. The systems may use row-column addressing in combination with bias activation to achieve an angular imaging aperture for ultrafast ultrasound imaging. Specifically, systems of the invention may employ row-column addressing to tune an imaging aperture.

Further, the methods of the invention may use bias voltage to tune the frequency range of the imaging array within a region of interest, with the sensitivity for a frequency range being driven by the bias voltage. Bias voltage may be used to deactivate imaging elements entirely in the region of interest or within the volume as a whole. Importantly, using bias voltage to activate imaging elements (i.e. cells) allows for imaging without multiplexing. Bias activation means that when a nominal bias voltage is applied to tune the imaging array to a target center frequency imaging will be activated on this row or column. Similarly, applying a bias voltage of OV or a voltage level where the sensitivity of the imaging element is minimal deactivates the imaging row or column. This allows for the enablement or disablement of full rows or columns on the array and provides for employing a flexible transmit/receive scheme on the activated row(s) and/or column(s) for electronic focusing.

In some embodiments of the methods, the controller is capable of controlling a rotary motor operably coupled to the ultrasound transducer unit to enable a continuous rotation or a positioning of the ultrasound transducer unit.

In some embodiments of the methods, the console may be configured to dynamically define and adjust the set of parameters during the course of one rotation such that the imaging characteristic and/or the image quality is optimized in a selected direction of the second selected region of interest as compared to the imaging characteristic and/or image quality within and/or outside the first selected region of interest.

Further, in some embodiments of the methods, the console is configured to dynamically define and adjust the set of parameters such that the imaging characteristic and/or the image quality is optimized over a continuous range of regions from the first selected region of interest to the second selected region of interest. The imaging characteristic and/or the image quality may be optimized, for example, via linear interpolation of the set of parameters over the range of regions from the first selected region of interest to the second selected region of interest.

As noted above, in some embodiments of the methods, console is further configured to synchronize a firing rate and a firing direction of the ultrasound transducer unit. The firing direction may be defined through a rotational position of the array, or in the context of a 2D flat/flexible array, through electronic control of the respective transducer area. The image characteristic in the second selected region of interest may be adjusted by dynamically adjusting a phase between at least a motor speed and a firing rate of the ultrasound transducer unit.

In some embodiments of the methods, the set of parameters may be dynamically adjusted by optimizing imaging depth and imaging resolution to provide a focused view of the first and/or second selected region of interest.

In some embodiments of the methods, the imaging depth is adapted in a region of interest. Changing the imaging depth may enable the creation of asymmetric imaging volumes around the circumference to selectively perform imaging of deeper structures in certain directions. The imaging depth may be kept constant in other directions where deeper tissue imaging is not important. This concept may be applied to electronic focusing/multiplexing as well as mechanical rotation. In some embodiments of the methods of the invention, the imaging resolution includes one or more of depth, lateral resolution, and angular resolution.

In some embodiments of the methods of the invention, the console is configured to adjust an imaging depth of the ultrasound transducer unit to thereby create an asymmetric imaging volume around the circumference to selectively perform imaging in a certain direction. As disclosed herein, the console may be configured to adjust a firing rate of the ultrasound transducer unit to thereby achieve a higher angular resolution in the selected region of interest.

Further, the console may be configured to adjust a planewave or diverging wave front characteristics and a steering angle of the ultrasound transducer unit to thereby achieve a complex shape of an imaging volume in the selected region of interest.

In some embodiments of methods of the invention, the planewave opening and/or the steering angle are adapted in a region of interest. Changing the planewave opening angle and steering angle yields complex shapes of the imaging volume that may be adapted to image a certain volume of interest. For example, in one direction, the planewaves may extend in a wide angle to achieve a wide opening angle in that direction while also having a narrow configuration in the other direction in order to achieve a higher imaging quality in that direction. This may be useful in intracardiac imaging when an intracardiac imaging catheter is placed in the right atrium to selectively provide deeper imaging with higher resolution in the septal region. For more lateral regions of this placement, for example the lungs, deeper penetration would not be required for anatomical context.

In some embodiments of methods of the invention, when good synchronization between the firing and the rotational position is achieved, selective image focusing may be achieved by configuring specific ultrasound firing patterns. The phase between the motor position and the firing sequence may be modified to control the direction of the “focus region”. If a non-rotating ultrasound design is used (e.g. in a 2D-matrix array system or any other form of 3D ultrasound system), firing and directional focusing/depth/resolution may be controlled entirely through electronic element activation and delays (electronic beamforming).

Some embodiments of the methods of the invention provide a mechanical solution for non-uniform rotation speed and firing rate. For example, instead of varying the firing rate, rotational speed may be varied by a mechanical change in the catheter to introduce friction inside the catheter in one direction. In some embodiments, a rotational velocity of the ultrasound transducer unit is varied by a mechanical change in the ultrasound transducer unit.

For example, for a rotating transducer with a constant firing rate, slowing down the rotation of the transducer using the mechanical change results in more firings from the transducer elements in the region in which the rotation is slowed. With a constant firing rate, this will result in more firings in the direction. For example, the mechanical change may be an area of eccentricity in the rotating transducer. The eccentricity may be a result of, for example, rubber or other material added to produce friction in the rotation of the transducer.

In some embodiments, the mechanical change introduces friction inside the ultrasound transducer unit in one direction to slow rotation of the ultrasound transducer unit. However, the systems also allows for compensating for mechanical behavior if friction is present inside the transducer unit such that it rotates slower in certain regions. Varying the firing rate in regions of interest or the volume as a whole compensates for this mechanical behavior. Thus, in some embodiments of methods of the invention, the firing rate is varied to compensate for friction present inside the ultrasound transducer unit. In some embodiments, the motor is non-discrete in order to change from one set of parameters to the next. In other embodiments of the system, the motor is a discrete function.

As noted above, the selected region of interest may be based on certain anatomical features. In some embodiments, the first and/or second selected region of interest is defined based on one or more anatomical features. The anatomical features may be, for example, a specific ablation path. Thus, the selected region of interest or focused region may be defined based on the anatomy imaged.

The methods of the invention provide for software-controlled adaptation of imaging parameters, for example, through software controlled focus-region steering. In any of the variations, the focus region may be freely chosen and adapted in software by adapting the phase between motor speed and repeating firing patterns, or by adapting the transmit/receive pattern. Adaptation may be performed dynamically during imaging.

The direction of the focus region may be controlled several ways. For example, the focus region may be directly exposed to the user via a user interface element, for example a direct control of the angle of the focus region, or by setting the angle of the focus region via indicating a specific region of interest in the image directly with the use of a mouse. The focus region may be implicitly controlled and/or adapted in combination with a 3D visualization of the data. In this way, the focus region will be implicitly tied to the region that is in the central focus of the 3D visualization. If the viewpoint of the 3D visualization changes, the imaging focus region is automatically adapted. This also takes into account knowledge about regions that are not displayed at all (outside of the current visualization field of view, or cropped away with a digital cropping function). The focus region may be automatically controlled by semantic analysis of the imaging data, to automatically detect particular anatomical regions and focus on these without user input, or by suggesting these regions to the user to focus on with a single interaction.

The console may include a computer program comprising an algorithm for evaluating, calculating, and optimizing the imaging parameters, as well as reconstructing the images using non-homogeneous spatial resolution and/or depth. As disclosed herein the imaging parameters may be, in non-limiting examples, planewave or diverging wave front characteristics. The planewave or diverging wave characteristics may be at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering. The imaging characteristics may be associated with, in non-limiting examples, at least one of an angular resolution, a field of view, an imaging depth, a transmit/receive pattern, a transmit firing rate, and a planewave opening angle.

Using defined algorithms, the console may be configured to provide feedback to an operator before or during operation of the imaging array for evaluating, calculating, and optimizing the imaging parameters. Thus, the systems provides for visualization of the reconstructed information assuming non-homogeneous spatial resolution or depth.

In some embodiments of the methods, the console is in active communication with a computing system comprising an algorithm for evaluating, calculating, and selectively optimizing the imaging parameters.

As previously noted, the transducer probe may be operably coupled to the console, which may generally control operation of the transducer probe (i.e., transmission of sound waves from the probe) and/or the controller. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

For example, in an exemplary embodiment, the console may generally include a computing device configured to communicate across a network. The computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for optimizing imaging parameters and subsequent reconstruction into 2D and/or 3D images of the non-homogenous data.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. The console may further provide control over an imaging assembly, including control over the emission of ultrasound pulses therefrom (intensity, frequency, duration, etc.) as well as control over the movement of the ultrasound transducer unit.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing selective image focusing, the system comprising:
a console configured to be operably associated with an ultrasound imaging device and exchange data therewith, wherein the console comprises a hardware processor coupled to non-transitory, computer-readable memory containing instructions executable by the processor to cause the console to:
define a set of parameters associated with operation of an ultrasound transducer unit of an ultrasound imaging device to achieve an imaging characteristic of one or more images captured via the ultrasound imaging device for a first selected region of interest; and
adjust the set of parameters, wherein adjusting the set of parameters provides directional focusing and/or imaging quality control at a second selected region of interest such that the imaging characteristic and/or an image quality is optimized for one or more directions at the second selected region of interest as compared to the first selected region of interest.

2. The system of claim 1, wherein the parameters comprise planewave or diverging wave front characteristics.

3. The system of claim 2, wherein the planewave or diverging wave characteristics comprise at least one of a virtual source focus, a distance, an opening angle, a steering angle, a number of individual firings, a transmit/receive pattern, a transmit firing rate, an imaging depth, a rotational velocity, a rotational position, and a 3-dimensional (3D) wave direction steering.

4. The system of claim 1, wherein the imaging characteristic is associated with at least one of an angular resolution, a field of view, an imaging depth, a transmit/receive pattern, a transmit firing rate, and a planewave opening angle.

5. The system of claim 1, wherein the console is configured to dynamically define and/or adjust the set of parameters.

6. The system of claim 5, wherein the imaging characteristic is selected and adjusted by dynamically adapting the transmit/receive pattern in the first and/or second selected region of interest.

7. The system of claim 6, wherein the first and/or second selected region of interest is defined based on one or more anatomical features.

8. The system of claim 5, wherein the set of parameters is dynamically adjusted by optimizing imaging depth and imaging resolution to provide a focused view of the first and/or second selected region of interest.

9. The system of claim 8, wherein the imaging resolution comprises one or more of depth, lateral resolution, and angular resolution.

10. The system of claim 2, further comprising an ultrasound imaging device operably coupled to the console, the ultrasound imaging device comprising an ultrasound transducer unit capable of full circumferential three-dimensional (3D) imaging.

11. The system of claim 10, wherein the console further comprises a controller to enable capture of planewave or diverging wave acquisition data over a circumferential 360-degree imaging region, wherein the first and/or second selected region of interest is within a circumference encompassing the 360-degree imaging region.

12. The system of claim 11, wherein the controller is capable of controlling a bias voltage selectively applied to one or more of a plurality of first and/or second electrodes of a transducer comprising an array of individual imaging elements, wherein the bias voltage defines a voltage for a row or a column connected to the electrode to activate or deactivate imaging by the individual imaging elements in the row or column to define an angular imaging aperture.

13. The system of claim 11, wherein the controller is capable of controlling a rotary motor operably coupled to the ultrasound transducer unit to enable a continuous rotation or a positioning of the ultrasound transducer unit.

14. The system of claim 10, wherein the console is configured to dynamically define and adjust the set of parameters during the course of one rotation such that the imaging characteristic and/or the image quality is optimized in a selected direction of the second selected region of interest as compared to the imaging characteristic and/or image quality within and/or outside the first selected region of interest.

15. The system of claim 10, wherein the console is configured to dynamically define and adjust the set of parameters such that the imaging characteristic and/or the image quality is optimized over a continuous range of regions from the first selected region of interest to the second selected region of interest.

16. The system of claim 15, wherein the imaging characteristic and/or the image quality is optimized via linear interpolation of the set of parameters over the range of regions from the first selected region of interest to the second selected region of interest.

17. The system of claim 10, wherein the console is further configured to synchronize a firing rate and a firing direction of the ultrasound transducer unit, and wherein the image characteristic in the second selected region of interest is adjusted by dynamically adjusting a phase between at least a motor speed and a firing rate of the ultrasound transducer unit.

18. The system of claim 10, wherein the console is configured to adjust an imaging depth of the ultrasound transducer unit to thereby create an asymmetric imaging volume around the circumference to selectively perform imaging in a certain direction.

19. The system of claim 10, wherein the console is configured to adjust a firing rate of the ultrasound transducer unit to thereby achieve a higher angular resolution in the selected region of interest.

20. The system of claim 10, wherein the console is configured to adjust a planewave or diverging wave front characteristics and a steering angle of the ultrasound transducer unit to thereby achieve a complex shape of an imaging volume in the selected region of interest.

21. The system of claim 10, wherein a rotational velocity of the ultrasound transducer unit is varied by a mechanical change in the ultrasound transducer unit.

22. The system of claim 21, wherein the mechanical change introduces friction inside the ultrasound transducer unit in one direction to slow rotation of the ultrasound transducer unit.

23. The system of claim 22, wherein the firing rate is varied to compensate for friction present inside the ultrasound transducer unit.
